(19) United States
(12) Patent Application Publication
(10) Pub. No.: US 2023/0417637 A1
(43) Pub. Date: Dec. 28, 2023

(54) UV COATINGS AND DYES FOR LASER CAPTURE MICRODISSECTION

(71) Applicant: George Mason Research Foundation, Inc., Fairfax, VA (US)

(72) Inventors: Lance A. LIOTTA, Bethesda, MD (US); Alessandra Luchini KUNKEL, Burke, VA (US); Marissa Ashton HOWARD, Richmond, VA (US); Amanda Haymond STILL, Manassas, VA (US); Harini SOMANCHI, Herndon, VA (US); Philip Andrew PAPPALARDO, Barryville, NY (US)

(73) Assignee: George Mason Research Foundation, INC., Fairfax, VA (US)

(21) Appl. No.: 18/036,137

(22) PCT Filed: Nov. 9, 2021
(86) PCT No.: PCT/US2021/058625

§ 371 Date: May 9, 2023

Related U.S. Application Data
(60) Provisional application No. 63/111,955, filed on Nov. 10, 2020.

(51) Int. Cl. G01N 1/28

(52) U.S. Cl. CPC G01N 1/2813; G01N 2001/284

(57) ABSTRACT

Laser Capture Microdissection devices and methods are provided. A Laser Capture Microdissection device contains a transmissive film and a transfer film. The transmissive film includes a first material configured to transmit laser energy. The transfer film includes a second material configured to absorb UV-laser energy. The transfer film contains an absorptive substance to absorb UV-laser energy and convert it into heat.

### CROSS REFERENCE TO RELATED APPLICATIONS

The present application claims the benefit of priority to U.S. provisional application No. 63/111,955 filed Nov. 10, 2020, the entire contents of which are incorporated herein by reference.

### FIELD OF THE INVENTION

This invention relates to LCM system compatible with UV radiation, which has a shorter wavelength compared to infra-red radiation. The invention is more particularly concerned to adapt LCM machines and consumables to be compatible with UV radiation.

### BACKGROUND OF INVENTION

Molecular analysis of cellular material is an essential dimension of diagnostic pathology that complements optical histologic analysis by the pathologist or scientist. The cells to analyzed include issue biopsy samples, mammalian or bacterial cell cultures, or bulk plant material, for which select subpopulations of cells may be isolated from the sample using microdissection methods such as Laser Capture Microdissection (“LCM”).

Laser Capture Microdissection (“LCM”) is an established technology used to isolate tumour cells, or other types of cells, from a heterogeneous piece of tissue under direct microscopic visualization. This is important because specific cell populations may have different properties than the tissue sample. For example, isolating just the tumor cells away from other immune cells in the same sample allows for more precise characterization of the molecular features of the tumor cells. The isolated cells are used in commercial diagnostic assays, clinical trials, and research studies by pharmaceutical companies and academic institutions. LCM is used by thousands of scientists worldwide.

The most popular and useful form of LCM employs a laser beam, or a source of radiation, to heat a thermopolymer cap that is held against the slice of tissue mounted on a glass slide. LCM machines utilize a beam of IR radiation to soften a capture surface of capture element, which melts onto the tissue underneath and allows certain cells to be plucked away from the rest of the tissue and analyzed separately. However, the wavelength of IR radiation is long, which means it can only be focused to micro dissect regions containing many cells.

FIG. 1A of U.S. Patent Publication No. 2017/0176301 (“the '301 publication”), illustrates a typical LCM system consisting of a thermoplastic film in contact with the tissue. The plastic film is uniformly impregnated with a dye that absorbs laser energy. The region of the plastic film positioned over the tissue region or cell of interest is selectively heated by the radiation causing this region to melt and embed itself into the tissue segment immediately underneath (FIG. 1B of the '301 publication). When the film is lifted off the tissue the portions of the tissue adherent to the undersurface of the film are ripped free of the tissue section (see e.g., Espina V., et al. (2006) Nature Prot. 1(2):586-603).

In addition to the instrument and method described in the 301 publication, other less popular methods and instruments for LCM are available, such as those from Leica or Zeiss (PALM). To simplifying discussion, mechanistic details of LCM will be described using the most popular instrument design as given in the 301 publication. However, “LCM” should be broadly understood to incorporate all commercially available LCM systems from diverse manufacturers.

Existing LCM technology, as described in the 301 publication, utilizes IR radiation to soften a thermopolymer layer of a capture device (also called as capture element), known as an LCM cap. This thermopolymer layer on the cap is impregnated with IR absorbing dye, such that when the IR passes through the cap, it is absorbed and converted to heat, melting the thermopolymer only in the regions irradiated. This allows for capture of selected tissue sections areas by irradiating only certain areas with the IR through the cap, while the cap is pressed to the surface of the tissue. While this system is highly effective, it is limited by the fact that the wavelength of IR radiation is long, thus putting a lower limit on the spot size possible for the IR beam. This lower limit dictates the size of the area to be micro-dissected; features smaller than the spot size of the beam cannot be isolated individually. The current LCM caps available are not transmissive to ultraviolet (UV). Application of UV to laser capture in the past has been severely limited as the plastic materials used for the components do not allow for the transmission of UV radiation.

Further in past UV-laser system has been limited due to “the presence of cells with UV-induced damage in the final cell population obtained. These UV-damaged cells are derived from the cells lying directly under the UV laser cutting path” (e.g., see Espina V., et al. (2006) Nature Prot. 1(2):586-603).

Therefore, there is long felt need for LCM system that has better precision and resolution compared to Infrared radiation-based LCM. Further, there is also a long felt need to develop a UV laser system such that the cell underlying directly under the UV laser cutting path is not damaged.

### SUMMARY OF INVENTION

LCM machines and consumables are adapted to be compatible with UV radiation, which has a shorter wavelength compared to infra-red radiation.

In an embodiment, a device includes a transmissive film and a transfer film, the transmissive film includes a first material configured to transmit a first ray containing a first wavelength of about 280 nm to about 380 nm, and the transfer film includes a second material configured to absorb the first ray; wherein the device is configured to be a capture element.

In an embodiment, the first material of device is configured to transmit about 80% of a ray with a wavelength of about 280 nm to about 1100 nm, and wherein the transfer film includes an absorptive substance.

In an embodiment, the absorptive substance includes a dye configured to absorb the first ray.

In an embodiment, the dye is configured to transmit a second ray with a second wavelength of about 380 nm to about 700 nm and absorb the first ray.

In an embodiment, the dye includes a broad band absorptive dye or a frequency specific absorptive dye.

In an embodiment, the absorptive substance includes a benzophenone dye, a benzotriazole dye or a triazine dye.

In an embodiment, the transfer film includes about 1 mM or less of the dye.

In an embodiment, the dye is configured to transmit about 80% of the second ray and absorb about 80% of the first ray.

In an embodiment, a method includes: a) placing a sample on a stage of an instrument having a capture element, wherein the instrument is configured to examine the sample; b) transmitting a UV ray through the capture element, wherein an absorptive substance in the capture element is configured to absorb the UV ray and convert into heat; c) capturing a portion of the sample on the capture element; and d) analysing the sample.

In an embodiment, the capture element includes a transmissive film and a transfer film, wherein the transmissive film is configured to transmit the UV ray and the transfer film includes a material and the absorptive substance.

In an embodiment, a slide includes the sample is configured to be placed on the stage of the instrument, wherein the slide includes a conductive substrate configured to activate the sample in subjection of the UV ray.

In an embodiment, the sample includes a label configured to be labile to the UV ray.

In an embodiment, the absorptive substance includes a benzophenone dye, a benzotriazole dye or a triazine dye.

In an embodiment, the sample further includes a molecular probe includes the label, wherein the UV ray reaching the sample dissociate the label from the molecular probe.

In an embodiment, the molecular probe includes an antibody, a peptide, or a small molecule.

In an embodiment, the label includes a DNA barcode, a heavy-metal label, a peptide label, or a small molecule label.

In an embodiment, a system includes a capture element includes a transmissive film and a transfer film, wherein the transmissive film includes a first material configured to transmit a ray containing a wavelength of about 300 nm to about 1100 nm, and the transfer film containing a second material configured to absorb a first ray containing of a wavelength of about 280 nm to 380 nm; wherein the second material includes an absorptive material and wherein the system is configured to examine a sample placed on a stage of the system.

In an embodiment, the transfer film further includes a third material configured to absorb a second ray with a second wavelength of about 700 nm to 1100 nm.

In an embodiment, the stage is configured to place on a slide containing the sample, wherein the slide includes a conductive substrate configured to activate the sample in subjection of the first ray.

In an embodiment, the absorptive material includes a benzophenone dye, a benzotriazole dye or a triazine dye.

### BRIEF DESCRIPTION OF THE FIGURES

FIG. 1A shows state-of-the-art laser capture microdissection systems.

FIG. 1B shows the addition of UV capture according to the present disclosure.

FIG. 2 shows the transmission spectra of Plexiglass G-UVT.

FIG. 3 shows the transmission spectra of several modified PMMA produce suitable for the capture material body.

FIG. 4 shows the structure of Benzophenone, Benzotriazole, and s-Traizine UV-absorbing dye scaffolds with one representative commercially available dye compound for each class shown

FIG. 5 shows the absorbance spectrum of octrizole (UV1), 2 t-Butyl-6(5-chloro-2H-benzo-triazole-2-yl)-4-methylphenol (UV2), and 2-(2-Hydroxy-5-methylphenol) benzotriazole (UV3), representatives of the triazole dye class. All have strong absorbances between 280-380 nm. All spectra depicted were measured at 0.1 mM in ethyl acetate.

FIG. 6 shows the absorbance spectrum of 2,2′,4,4′-tetrahydroxybenzophenone (UV4), and 2, 4-dihydroxybenzophenone (UV5), representatives of the benzophenone dye class. All have strong absorbances between 280-380 nm. All spectra depicted were measured at 0.1 mM in ethanol.

FIG. 7 is a cell capture element description. Region A is the capture element body, while region B is the capture element thermopolymer transfer film.

FIG. 8 shows use of high and low settings either for cell capture or for label dissociation.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that includes a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” and “about” can, in some embodiments, mean within plus or minus ten percent of the stated value. In other embodiments, “approximately” and “about” can mean within plus or minus five percent of the stated value. In further embodiments, “approximately” and “about” can mean within plus or minus three percent of the stated value. In yet other embodiments, “approximately” and “about” can mean within plus or minus one percent of the stated value.

As used herein, the terms “optional” or “optionally” mean that the subsequently described event or circumstance may or may not occur, and that the description includes instances where said event or circumstance occurs and instances where it does not.

The word ‘or’ as used herein means any one member of a particular list and also includes any combination of members of that list.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

Laser capture microdissection (LCM) technique is a sample preparation technique that enables isolation of specific cells from a mixed population under microscopic visualization. This technique of isolating a pure sample from a heterogeneous mixture allows for more efficient and accurate results with downstream micro genomics applications such as next-generation sequencing, Sanger sequencing, PCR, and proteomics etc.

The laser capture operation can be simply described as firstly the microscope stage or platform is centered, and the transfer film coupled to cap or capture element is placed in position above the tissue sample on the slide. The transfer film contacts the tissue sample. Then, the laser activates the transfer film, which absorbs energy from the laser that melts a small area of the cap to fuse the cap to selected areas in the tissue, forming a spot. The selected portion of sample can be used for further processing and analysis.

Alternatively, the transfer film is spaced from the sample (noncontact laser micro capture). In this case, the expansion of the transfer film upon activation by the laser pulse will simply inject a portion of the transfer film into the tissue sample for capturing target sample cells.

The terms and phares used in this application following definitions:
“UV or Ultra-violet” as used herein is defined as a form of electromagnetic radiation with wavelength from 10 nm (with a corresponding frequency around 30 PHz) to 400 nm (750 THz), shorter than that of visible light, but longer than X-rays.
“UV lasers” are defined as lasers emitting UV wavelengths such as but not limited to gas lasers, laser diodes, and solid-state lasers manufactured to emit ultraviolet rays. The nitrogen gas laser uses electronic excitation of nitrogen molecules to emit a beam that is mostly UV. Another type of high-power gas laser is excimer lasers. They are widely used lasers emitting in ultraviolet and vacuum ultraviolet wavelength ranges. Presently, UV argon-fluoride excimer lasers operating at 193 nm are routinely used in integrated circuit production by photolithography. Direct UV-emitting laser diodes are available at 375 nm. Any known UV laser can be used in the present invention.
“Capture element” as used herein is defined as a device that enables the extraction and detection of biological molecules from small numbers of cells, tissue, or any other sample. Capture element can also be defined as a device with the function like LCM cap. Capture element is also written as a capture device. The capture element can capture cells from the tissue sample, therefore it also called as cell capture element.
“Capture” as used herein is defined as a process to extract the desired portion from the sample that is examined under instruments such as LCM.
“Sample” as used herein is used in its broadest sense and includes environmental and biological samples. Environmental samples include material from the environment such as soil and water. Biological samples may be animal, including, human, fluid (e.g., blood, plasma, serum, urine, saliva), solid (e.g., stool), tissue, liquid foods (e.g., milk), and solid foods (e.g., vegetables). For example, a pulmonary sample may be collected by bronchoalveolar lavage (BAL), which includes fluid and cells derived from lung tissues. Other examples of biological samples may include a cell, tissue extract, body fluid, chromosomes or extrachromosomal elements isolated from a cell, genomic DNA, RNA, cDNA and the like. Samples may also include tissue sample
“Tissue sample” as used herein is a piece of tissue removed from an organism, e.g. a biopsy, or a whole organ of an organism or even a whole organism.
“UV-permissible base material” as used herein is defined as a material that allow transmission of UV light.
“Analysis” as used herein is defined as a process governed by one or various techniques for analysis and identification of molecules such as nucleic acids, amino acids, peptides, proteins, polymers or biological markers in the genome and proteome as known to a person skilled in the art. For example, but not limited to proteomic analysis such as protein profiling, genetic analysis such as individual's genetic code and how their cells express their genes as protein, RNA profiling etc. In some embodiment, molecular analysis may refer to identification of cell type in a tissue sample. Molecular analysis is also defined as molecular profiling.
“Transmission” as used herein is defined as a process of to allow travel of an electromagnetic ray such as UV ray, IR ray, visible ray or any other electromagnetic ray through a medium such as but not limited to glass or plastic.
“Transmissive film” as used herein is defined as a material that allow transmission of ray of desired wavelength. For example: UV-transmissive film allows transmission of UV ray.
“Absorptive substance” as used herein is defined as a material that absorbs the ray of desired wavelength and converts the energy of electromagnetic ray into heat.
“Spot size” can be broadly defined as a radius of beam of ray. The UV has shorter wavelength compared to visible or infrared radiation, therefore the spot size formed by a UV ray would be shorter than visible and infra-red radiation.
“Transfer layer” can be defined as a low temperature melting thermopolymer with a large volume increase associated with the phase transition from a solid to a liquid (e.g., ethylene vinyl acetate) which can be dyed so as to couple to radiation from laser of a specific frequency. When the desired part in sample is identified for LCM, the laser is activated, and transfer layer activated to adhere to sample at the selected region. At many places in description, transfer layer is also written as thermopolymer layer.
“Activate the sample” as used herein is defined as making conditions favorable for capture portion of sample to be further analyzed using various analysis method.

Existing LCM technology, utilizes infrared radiation (IR) radiation to soften a thermopolymer layer of a capture device, known as an LCM cap. This thermopolymer layer on the cap is impregnated with IR absorbing dye, such that when the IR passes through the cap, it is absorbed and converted to heat, melting the thermopolymer only in the regions irradiated. This allows for capture of selected tissue sections areas within the sample by irradiating only certain areas with the IR through the cap, while the cap is pressed to the surface of the tissue. This system is limited by the fact that the wavelength of IR radiation is long, thus putting a lower limit on the spot size possible for the IR beam. This lower limit dictates the size of the area to be micro-dissected; features smaller than the spot size of the beam cannot be isolated individually

In present invention, LCM machines and consumables such as capture elements, dyes, etc. are compatible with UV radiation, which has a shorter wavelength compared to infra-red radiation. Application of UV in laser capture in the past has been severely limited as the plastic materials used for the components of LCM do not allow for the transmission of UV radiation.

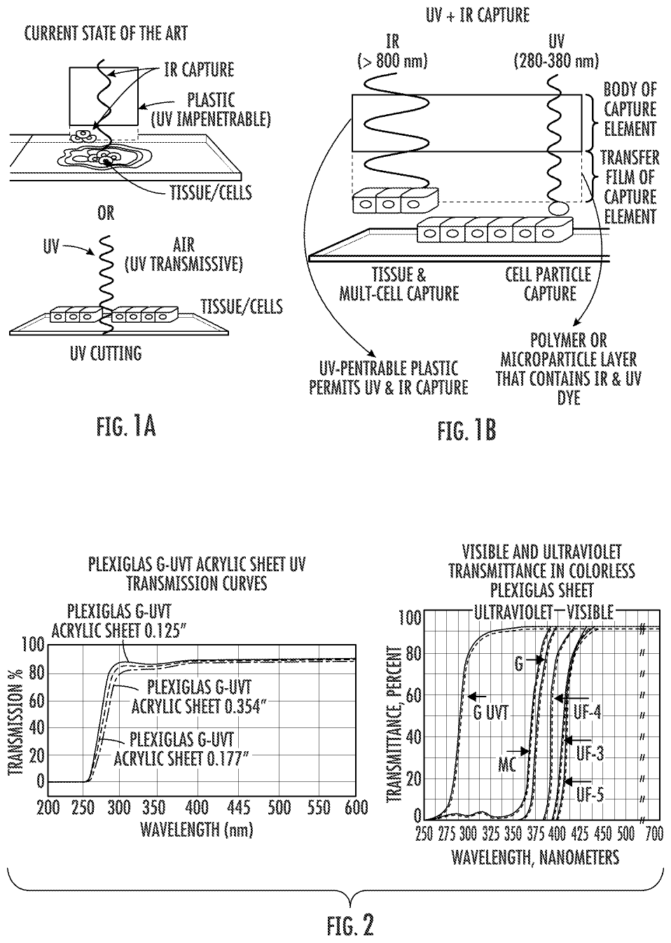

FIGS. 1A-B show a contrast between existing technology and the present invention which incorporates a UV capture component into the LCM system. In an embodiment, combining both IR and UV capture allows for the highest flexibility for the customer to capture large sections of tissue or single cells.

In an embodiment, the LCM of present invention has modifications to components of the system, primarily the capture device, as it now incorporates different dyes at different concentrations and includes a UV-permissible base material.

In an embodiment, the use of a UV laser is the source of radiation, and the use of dyes to absorb UV ray (UV-dye) in the capture step. The capture device incorporates different dyes at different concentrations and includes a UV-permissible base material.

In an embodiment, UV lasers in LCM systems has UV-transmissive film for LCM components and UV dye-impregnated transfer film for additional flexibility for use with UV lasers in LCM systems.

In an embodiment, UV lasers in LCM systems has UV-transmissive film for LCM components and UV-absorbing coated slides and UV dye-impregnated transfer film for additional flexibility for use with UV lasers in LCM systems.

The present invention is an improvement to existing technology because they allow for microdissection with smaller spot size leading to improved precision. The spot size measured by the present invention is less than 10 μm, less than 9 μm, less than 8 μm, less than 7 μm, less than 6 μm, less than 5 μm, less than 4 μm, less than 3 μm, less than 2 μm, less than 1 μm, less than 0.5 μm, less than 0.1 μm or less.

In an embodiment, the UV pass through a cell capture element, which includes both a transmissive film as well as a thermopolymer film containing the absorptive substances. Few examples of absorptive substances containing dyes are shown in FIG. 7.

The transmissive film as used herein is a film that allows transmission of visible light in the wavelength range of about 380 to about 780 nm, infrared radiation (IR) radiation in the wavelength range of about 700 to about 1100 nm and UV radiation in the wavelength range from about 300 to about 380 nm.

In an embodiment, the transmissive film has greater than about 80% transmission of UV radiation in the wavelength range from about 300 to about 380 nm, greater than about 80% transmission of visible light in the wavelength range of about 380 to about 780 nm, and greater than about 80% transmission of near infrared radiation about 700 to about 1100 nm.

In an embodiment, the transmissive film has greater than about 90% transmission of UV radiation in the wavelength range from about 300 to about 380 nm, greater than about 90% transmission of visible light in the wavelength range of about 380 to about 780 nm, and greater than about 90% transmission of near infrared radiation about 700 to about 1100 nm.

In an embodiment, the transmissive film has greater than about 95% transmission of UV radiation in the wavelength range from about 300 to about 380 nm, greater than about 95% transmission of visible light in the wavelength range of about 380 to about 780 nm, and greater than about 95% transmission of near infrared radiation about 700 to about 1100 nm.

In an embodiment, the body of capture element is made up of transmissive film.

In an embodiment, the body of the capture element could be composed of Plexiglass G-UVT plastic.

In an embodiment, the body of the capture material could include another brand's version of UV-penetrable poly methyl methacrylate (PMMA) other than that available from Plexiglass, such as Lucite Perspex XTOX02.

In an embodiment, transmission spectra of Plexiglass G-UVT is shown in FIG. 2.

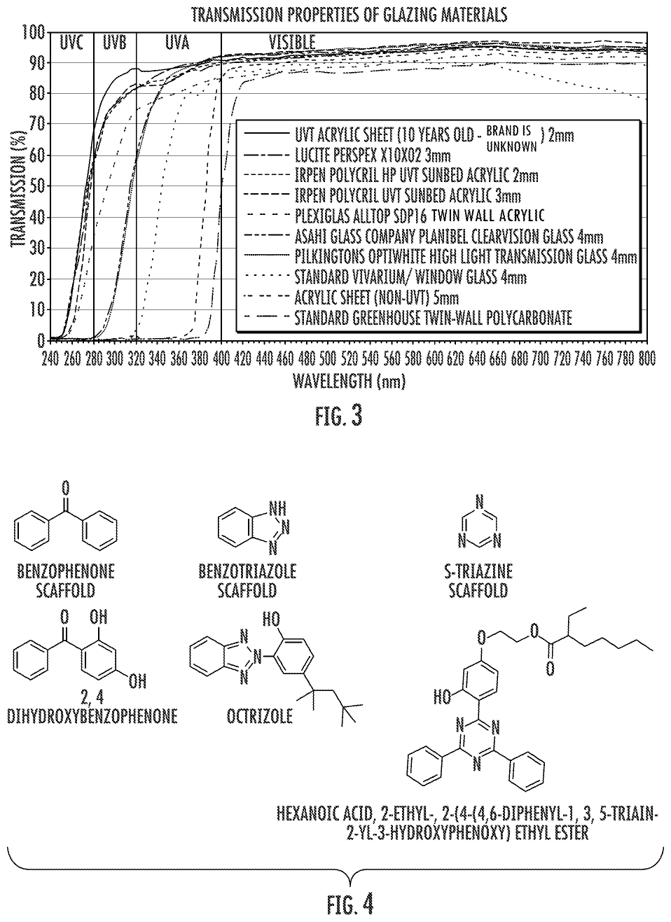

In another embodiment, transmission spectra for other potentially appropriate materials are shown in FIG. 3, UVT (white), Lucite (red), Policril HP UVT (orange) and Policril UVT (yellow). In FIG. 3, the order of the glazing materials from the highest to the lowest point on the y-axis (transmission %) between 495-510 mm corresponds to Pilkington's Optiwhite high light transmission glass (4 mm), Irpen Polycril UVT sunbed acrylic 3 mm, Irpen polycril HP UVT sunbed acrylic 2 mm, UVT acrylic sheet (10 years old, brand unknown, 2 mm), Asahi Glass Company Planibel Clearvision glass 4 mm, Lucite Perspex XTOX02 3 mm, acrylic sheet (non-UVT) 5 mm, standard vivarium/window glass 4 mm, Plexiglas all-top SDP-16 twin-wall acrylic, and standard greenhouse twin-wall polycarbonate.

The thermopolymer film as used herein is a film made of polymer material that becomes pliable or moldable at a certain elevated temperature. The thermopolymer is also called as thermoplastic in this specification. A variety of thermoplastic polymer films is widely used as heat activated adhesives that are suitable for the transfer film. In one embodiment, it is preferable to use a polymer film having a high melt index range such as greater than 100 dg/min, so that it is activatable at lower temperatures to avoid damage to or change in the nature of the tissue sample.

In one embodiment, the thermopolymer polymer film having a high melt index range such as greater than 100 dg/min, so that it is activated at temperature ranging 50° C.-150° C.

In one embodiment, the thermopolymer film is made up of ethylene vinyl acetate (EVA).

In one embodiment, LCM can be employed using a patterned (e.g., micropatterned) transfer film contains projections, such as micropillars, micro projections, hydrogel microspheres, and/or microneedles. The micropatterned transfer film is disclosed in US 2017/0176301, the entire contents of which are incorporated herein by reference for the devices and methods disclosed therein.

In an embodiment, the micropatterned thermoplastic film, such as an EVA film, used in LCM techniques can include an absorptive substance.

In an embodiment, the capture element has a foot made up of thermopolymer film.

The thermopolymer film may be impregnated with different dyes that absorb UV radiation. By combining dyes of slightly different lambda max absorbance values within the cap, absorption of the incoming UV radiation may be controlled, allowing for control over the selective swelling and melting of the cap “feet” as they make contact and become stuck to the tissue surface and/or cells of interest.

To enhance energy absorption, the transfer film can include an absorptive substance. There are many well-known absorptive substances that are capable of being thermally coupled to the transfer film. For example, the absorptive substance can include an absorptive dye or simply called as dye in this specification.

In an embodiment, the dye can be either a broadband absorptive dye or a frequency-specific absorptive dye. A broadband absorptive dye is one capable of absorbing a portion of the electromagnetic spectrum. A UV-broad band absorptive dye would have capacity to absorb the ray within the range of from 10 nm to 400 nm, preferably within 50 nm to 400 nm, further preferably within 100 nm to 400 nm. Similarly, IR-broad band absorptive dye would have capacity to absorb the ray within the wavelength range of infra-red radiation. The broadband absorptive dye can have a relatively broad absorption line or absorb energy throughout the ultraviolet region of the spectrum.

In an embodiment, absorptive dye can be an ultraviolet-absorbing benzophenone dye, benzotriazole dye, or triazine dye. An absorptive substance can have a strong absorption in the 280-380 nm region, a wavelength region that overlaps with laser emitters used to selectively melt the film or cut tissue. The absorptive substance is mixed with the melted bulk plastic at an elevated temperature, thermoplastic includes the absorptive substance is then manufactured into a film using standard film manufacturing techniques.

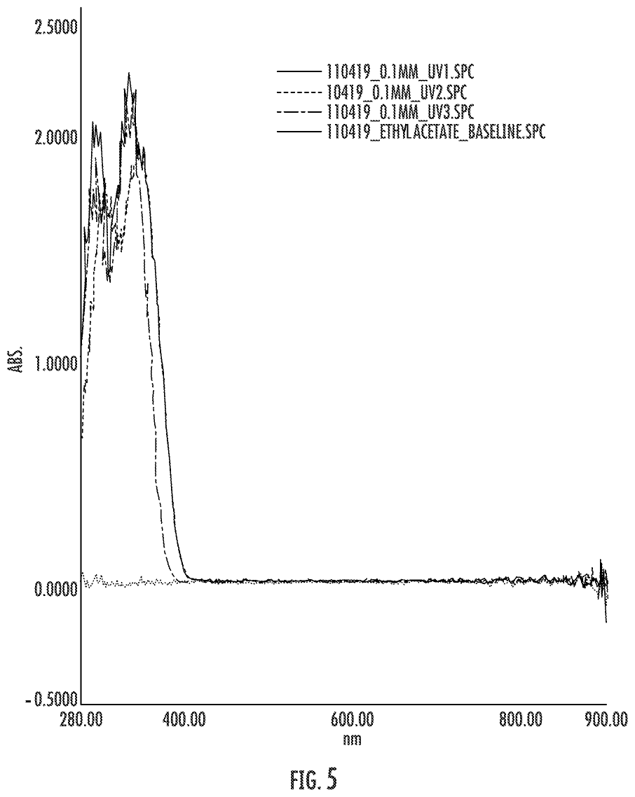

In an embodiment, UV-absorbing dyes including benzotriazole (scaffold for benzotriazole dyes, once such dye octriazole), s-triazine (scaffold for striazine dyes, one such Hexanoic acid, 2-ethyl-,2-(4-(4,6-diphenyl-1,3,5-triain-2-yl-3-hydroxylphenoxy) ethyl ester shown), and benzophenone (scaffold for benzophenone dyes, one such 2,4 Dihydroxybenzophenone). The structures of candidate UV-absorbing dyes are shown in FIG. 4.

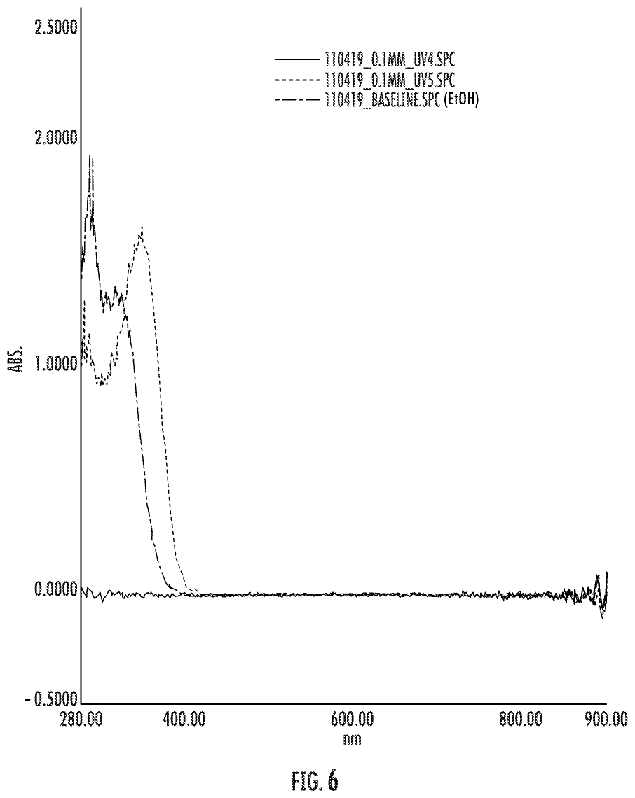

In another embodiment, the absorbance spectrum of the benzophenone, benzotriazole, and s-triazine based dyes show strong absorbances at or near 340 nm and can be used singly or in conjunction to cover the UV range from 300-380 nm. The absorption spectra are shown in FIG. 5 and FIG. 6. In FIG. 5, at the beginning of the x-axis, in order from the greatest value on the y-axis to the lowest value on the y-axis, the spectra correspond to UV1, UV3, and UV2 as described above, and an ethyl acetate baseline. In FIG. 6, at the beginning of the x-axis, in order from the greatest value on the y-axis to the lowest value on the y-axis, the spectra correspond to UV5 and UV4 as described above, and a baseline.

In an embodiment, dye concentration in the thermopolymer can be about 1 mM, about 0.5 mN or less down to 0.1 mM.

In another embodiment, the dye concentration in the UV cap could be high, such that all incident UV light from the laser capture microdissection instrument is absorbed and converted into heat, melting the thermopolymer layer of the laser capture microdissection cap and allowing the polymer to melt onto the tissue surface. In this embodiment, a select region of interest is attached the melted polymer and can be plucked from the bulk tissue for downstream molecular analysis.

An embodiment, the dye concentration in the UV cap could be low, such that a small amount of incident UV light from the laser capture microdissection instrument is not absorbed by the cap but transferred to the tissue surface. The remainder of the incident UV light absorbed by the LCM cap is converted to heat, melting the thermopolymer layer of the laser capture microdissection cap and allowing the polymer to melt onto the tissue surface. In this embodiment, a select region of interest is attached the melted polymer and can be plucked from the bulk tissue.

Yet another embodiment, the absorptive substance can be applied directly, with or without mild heat, to the preformed micropatterned surface of the LCM cap.

In this embodiment, the small amount of UV light that reaches the tissue surface could dissociate a UV-labile label from a molecular probe that is added to the surface of the tissue prior to microdissection. In this embodiment, the laser capture microdissection process both liberates a tissue section of interest and liberates a label from the molecular probe that can then be analyzed for presence or identity.

Molecular probe as used herein is defined broadly as a group of atoms or molecules used in molecular biology or chemistry to study the properties of other molecules or structures. Different molecular probes are disclosed in an article molecular probes and their application in International Journal of Lifesc. BT. Pharm., 2, 32-42 published in year 2013, which is incorporated herein in its entirety. Probe used are double and single standard DNAs, mRNAs, and other RNAs synthesized in vitro, cDNA probes, synthetic Oligonucleotide probes, Radiolabeled Probes, 3H, 32P, 35s,14c,¹²⁵1, 4-(Phenoxymethyl)piperidine, Biotin, Digoxigenin, Digoxigenin-II-dUTP, heterologous cDNA probes, Kinetoplast DNA (KDNA, a repetitive DNA sequence), Kinetoplast DNA (KDNA) a repetitive DNA, total parasite DNA, Ribosomal RNA gene, Repetitive DNA sequences, Ribosomal DNA, Ribosomal DNA.

Yet another embodiment, the molecular probe could be but is not limited to an antibody, peptide, or small molecule labelled with a UV-labile labeling element.

Yet another embodiment, the labeling element could be but is not limited to a DNA barcode, a heavy-metal label, a peptide label, or a small molecule label. Labelling refers to the use of the appropriate molecular labels to detect or purify the labelled protein and its binding partners. The DNA barcode is a short section of DNA from a specific gene or genes used to identify unknown gene or sequence.

In an embodiment, the present invention capture element allows both IR and UV transfer layer. The IR transfer layer would absorb the IR ray and capture the selected region from the sample. The UV transfer would absorb UV rays and capture smaller section compared to IR. This would provide larger flexibility to user

In an embodiment, combining both IR and UV capture allows for the highest flexibility for the customer to capture large sections of tissue or single cells.

In an embodiment, the body of capture element allows UV as well as infrared and visible light transmission. This allows a user to microdissect smaller regions containing many cell to single cell or less. Being able to analyze single cells will allow for greater scientific research into several conditions in which single cells of interest may carry a lot of information about the state of the disease or condition in that tissue section.

The invention can be used a) to perform high resolution procurement of tissue cells orb) to perform high resolution capture of molecular probes away from the tissue.

The capture element can be a laser capture microdissection cap. In one embodiment, the body of the capture element could be formed into a sheet or a slide.

In an embodiment, the slide is coated with UV absorbing substance or a coating.

In an embodiment, the coated slide and/or the UV-dye impregnated EVA transfer film, the UV-absorbing compound have the following qualities: the transparent coating or dye can be a metallic polymeric or organic coating or dye with the property that it is transparent and can be melted, softened, or caused to absorb UV-light by subjection to UV laser light found in an LCM system.

In an embodiment, the coating or dye will contain a less than 1000 kDa molecule that has a transmittance >80% in the visible wavelength range (380-700 nm) and an absorbance of >80% in the ultraviolet wavelength range (280-380 nm, UV-A & UV-B).

In another embodiment, the surface of the glass slide substratum may be treated by coating the surface with a transparent or semi-transparent conductive substrate that will melts or softens under subjection to the UV laser beam of the LCM machine (i.e., when irradiated with electromagnetic energy or wavelengths in the UV or IR spectrum). This transparent, conductive coating can be a metallic, polymeric, or organic coating with the property that it is transparent and can be melted or softened by subjection to UV laser light of the appropriate wavelength. This allows the glass slide substratum to easily dissociate from the tissue section of interest, improving capture efficiency of LCM.

In another embodiment, the surface of the glass slide substratum may be treated by coating the surface with a transparent or semi-transparent absorptive substrate that will melts or softens under subjection to the UV laser beam of the LCM machine (i.e., when irradiated with electromagnetic energy or wavelengths in the UV or IR spectrum). This transparent, conductive coating can be a metallic, polymeric, or organic coating with the property that it is transparent and can be melted or softened by subjection to UV laser light of the appropriate wavelength.

The appropriate wavelength for UV laser beam varies from 10 nm to 400 nm, preferably to 200 nm to 400 nm. This allows the glass slide substratum to easily dissociate from the tissue section of interest, improving capture efficiency of LCM.

In another embodiment, the surface of the glass slide substratum may be treated by coating the surface with a transparent or semi-transparent absorptive substrate that will melts or softens under subjection to the IR laser beam of the LCM machine (i.e., when irradiated with electromagnetic energy or wavelengths in the UV or IR spectrum). This transparent, conductive coating can be a metallic, polymeric, or organic coating with the property that it is transparent and can be melted or softened by subjection to IR laser light of the appropriate wavelength. This allows the glass slide substratum to easily dissociate from the tissue section of interest, improving capture efficiency of LCM.

By incorporating an additional coating on the glass slides, such embodiments effectively ameliorate the concern that the affinity of the tissue section of interest for the glass slide is too great to be completely overcome by the affinity of the LCM cap to the tissue section, which may otherwise contribute to tearing or incomplete capture of the cells of interest.

In another aspect, the slides which are used in LCM according to some embodiments were treated with different material from absorptive material of transfer layer to evaluate their effects on capture efficiency without sample distortion. Coated slides were compared for capture efficiency versus cells microdissected from conventional silicon dioxide uncoated microscope slides.

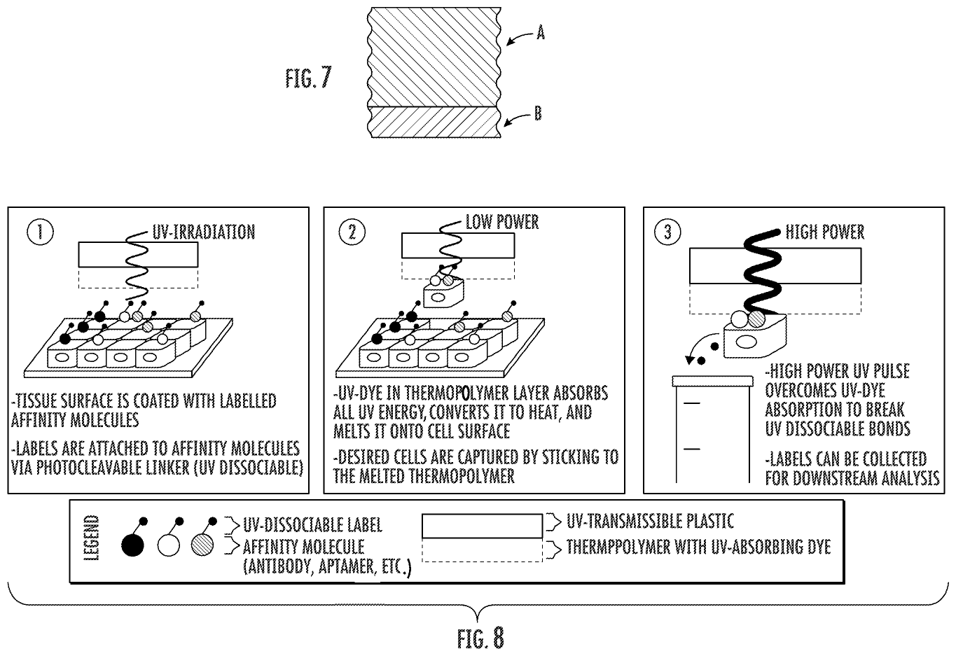

In an embodiment, the use of UV in LCM opens additional possibilities for labelling and detection of specific molecular tissue features. FIG. 8 outlines a process, in which low power UV power settings (or power settings under the absorptive capacity of the UV dye in the capture element), allow for melting of the thermopolymer layer, leading to cell capture. As shown in FIG. 8 in section (1) (left portion), the tissue surface is coated with labelled affinity molecules. Labels are attached to affinity molecules via photocleavable linker (UV-dissociable). In FIG. 8, section (2) (middle portion), UC-dye in thermopolymer layer absorbs all UV energy, converts it to heat, and melts it onto cell surface. Desired cells are captured by sticking to the melted thermopolymer. In FIG. 8, section (3) (right portion), a high-power UV pulse overcomes UV-dye absorption to break UV-dissociable bonds. Labels can be collected for downstream analysis.

If desired, a subsequent higher power setting post capture can be used in which the power setting exceeds the absorptive capacity of the capture element, allowing a small amount of UV to penetrate through the thermopolymer layer to the captured tissue surface, where it can be used to dissociate labels from cell markers if desired.

In an embodiment, the present invention is not obvious because it allows UV as well as infrared and visible light transmission in this major advance. This allow a user to microdissect smaller regions containing many cells to a single cell or less. Being able to analyze single cells will allow for greater scientific research into several conditions in which single cells of interest may carry a lot of information about the state of the disease or condition in that tissue section. The invention can be used a) to perform high resolution procurement of tissue cells or b) to perform high resolution capture of molecular probes away from the tissue. This invention disclosure for described the new plastic material itself, as well as other components needed for updated consumables as well as previously impossible applications enabled by this invention.

All references, including granted patents and patent application publications, referred herein are incorporated herein by reference in their entirety.

### REFERENCES

1) Vasavirama, K. (2013). Molecular probes and their applications. Int J Lifesc Bt Pharm Res, 2, 32-42.
2) U.S. Patent Publication No. 2017/0176301.
3) Espina V., et al. (2006) Nature Prot. 1(2), 586-603.
4) Michael, R. et. al (1996). Laser Capture Microdissection, Science, Volume 274, Number 5289, (8), 998-1001.

What is claimed is:

1. A device comprising a transmissive film and a transfer film, the transmissive film comprising a first material configured to transmit a first ray comprising a first wavelength of about 280 nm to about 380 nm, and the transfer film comprising a second material configured to absorb the first ray; and wherein the device is configured to be a capture element.

2. The device of claim 1, wherein the first material is configured to transmit about 80% of a ray with a wavelength of about 280 nm to about 1100 nm, and wherein the transfer film comprising an absorptive substance.

3. The device of claim 2, wherein the absorptive substance comprises a dye configured to absorb the first ray.

4. The device of claim 3, wherein the dye is configured to transmit a second ray with a second wavelength of about 380 nm to about 700 nm and absorb the first ray.

5. The device of claim 3, wherein the dye comprises a broad band absorptive dye or a frequency specific absorptive dye.

6. The device of claim 3, wherein the absorptive substance comprises a benzophenone dye, a benzotriazole dye or a triazine dye.

7. The device of claim 3, wherein the transfer film comprises about 1 mM or less of the dye.

8. The device of claim 4, wherein the dye is configured to transmit about 80% of the second ray and absorb about 80% of the first ray.

9. A method comprising:
a) placing a sample on a stage of an instrument comprising a capture element, wherein the instrument is configured to examine the sample;
b) transmitting a UV ray through the capture element, wherein an absorptive substance in the capture element is configured to absorb the UV ray and convert into heat;
c) capturing a portion of the sample on the capture element; and
d) analyzing the sample.

10. The method of claim 9, wherein the capture element comprises a transmissive film and a transfer film, wherein the transmissive film is configured to transmit the UV ray and the transfer film comprising a material and the absorptive substance.

11. The method of claim 9, wherein a slide comprising the sample is configured to be placed on the stage of the instrument, wherein the slide comprises a conductive substrate configured to activate the sample in subjection of the UV ray.

12. The method of claim 9, wherein the sample comprises a label configured to be labile to the UV ray.

13. The method of claim 9, wherein the absorptive substance comprises a benzophenone dye, a benzotriazole dye or a triazine dye.

14. The method of claim 12, wherein the sample further comprises a molecular probe comprising the label, wherein the UV ray reaching the sample dissociate the label from the molecular probe.

15. The method of claim 14, wherein the molecular probe comprises an antibody, a peptide, or a small molecule.

16. The method of claim 12, wherein the label comprises a DNA barcode, a heavy-metal label, a peptide label, or a small molecule label.

17. A system comprising a capture element comprising a transmissive film and a transfer film, wherein the transmissive film comprising a first material configured to transmit a ray comprising a wavelength of about 300 nm to about 1100 nm, and the transfer film comprising a second material configured to absorb a first ray comprising of a wavelength of about 280 nm to 380 nm; wherein the second material comprises an absorptive material and wherein the system is configured to examine a sample placed on a stage of the system.

18. The system of claim 17, wherein the transfer film further comprises a third material configured to absorb a second ray with a second wavelength of about 700 nm to 1100 nm.

19. The system of claim 17, wherein the stage is configured to place on a slide comprising the sample, wherein the slide comprises a conductive substrate configured to activate the sample in subjection of the first ray.

20. The system of claim 17, wherein the absorptive material comprises a benzophenone dye, a benzotriazole dye or a triazine dye.
